(19) United States
(12) Patent Application Publication
(10) Pub. No.: US 2010/0036246 A1
(43) Pub. Date: Feb. 11, 2010

(54) Automatic fat thickness measurements

(76) Inventors: Leonid Kushculey, Rehovot (IL); Vladimir Goland, Ashdod (IL)

Correspondence Address: EITAN MEHULAL LAW GROUP, 10 Abba Eban Blvd. PO Box 2081, Herzlia, 46120, IL

(21) Appl. No.: 12/222,334

(22) Filed: Aug. 7, 2008

(51) Int. Cl. A61N 7/00; A61B 8/00

(52) U.S. Cl. 600/439; 601/2

(57) ABSTRACT

There is provided a system for lysing of adipose tissue that includes a fat measuring device adapted to automatically measure thickness of a fat layer in a target area of a subject, a transducer adapted to transmit focused ultrasound and a controller adapted to receive a measurement from said fat measuring device and to trigger said transducer to transmit focused ultrasound to said fat layer.

### BACKGROUND

A popular area in the field of aesthetic medicine is the removal of subcutaneous fat and the reduction of the volume of adipose tissue and/or cellulite, resulting in the reshaping of body parts, frequently referred to as “body contouring”.

One such technique is a non-invasive ultrasound-based procedure for fat, cellulite and adipose tissue removal. The treatment is based on the application of focused therapeutic ultrasound that selectively targets and disrupts fat cells without damaging neighboring structures. This may be achieved by, for example, a device, such as a transducer, that delivers focused ultrasound energy to the subcutaneous fat layer.

For such medical and cosmetic purposes, it is often desirable to be able to focus the ultrasonic output of the transducer. To achieve this, transducers may include various shapes and sizes. For example, the transducer may be comprised of a cup-shaped piezoelectric ceramic shell with conductive layers forming a pair of electrodes covering the convex outside and concave inside of the piezoelectric shell. Occasionally, such transducers are hemispherical, with the “open end”, that is, the equatorial plane being positioned toward the subject being treated. In addition, the transducer may further include more than one transducing region such that an effect of dynamic focusing may be achieved by the use of phased array transducers.

The transducer is generally excited to vibrate and generate ultrasound by pulsing it using a high frequency power supply generally operating at a resonant frequency of vibration of the piezoelectric material.

When using a hemispherical transducer, it exhibits an “axial focal pattern”. This is an ellipsoidal pattern having a relatively small cross section and a relatively longer axis coincident with a “longitudinal” axis of the transducer, that is, a line through the center of rotation of the transducer perpendicular to the equatorial plane.

Specific, pre-set ultrasound parameters are used in an attempt to ensure that only the fat cells within the treatment area are targeted and that neighboring structures such as blood vessels, nerves, muscles and connective tissue remain intact. Such parameters may include frequency, intensity, average acoustic power, and the like. In addition, it is desired to position the focal area of the transducer in the correct location, within the adipose tissue layer/volume.

Thus, to allow the optimal effectiveness of the treatment with maximal safety and to allow ease of operation such that even inexperienced care providers are able to easily and effectively determine the correct location of position of the focal area of the transducer, an automatic determination of fat cells and fat tissue (adipose tissue) from the neighboring structures would be beneficial.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there is provided a method for automatic fat thickness measurement that is adapted to automatically calculate the thickness of fat tissue layer in a target area of a subject body. There is further provided a device and system for automatic fat thickness measurement that may provide a caregiver with real-time measurements of the fat thickness in the target area to be treated. There is further provided a device and system for lysing fat tissue that includes measuring fat thickness layer in a target area and focusing ultrasonic energy to the fat layer in the target area. The fat measuring device may further be adapted to determine fat thickness in the target area, by implementing a method for automatic fat thickness measurement. The fat measuring device may further be associated with an ultrasonic transducer that is adapted to provide ultrasonic energy to the target area of a body of a subject. The fat measuring device may further interact with the controller of the ultrasonic transducer to optionally adjust automatically and/or manually the operating parameters and/or mode of operation of the transducer so as to allow the caregiver to use the ultrasonic transducer in body contouring treatment, such that the treatment is performed at the most accurate and effective depth of treatment, so that fat tissue is specifically targeted by the ultrasonic treatment.

According to further embodiments, the use of automatic fat measuring may ensure, in real time, that the ultrasonic treatment is performed within the desired target tissue/volume (such as, for example, adipose tissue), and that the operator of the transducer (such as, for example, a care giver) is not involved in the process of the decision making, thus making the ultrasonic treatment “operator independent”. To this aim, an automatic fat measurement system, which may be used to ensure that the ultrasonic system operating parameters/mode of operation (such as, for example, focal depth, spatial position, and the like) are set up optimally, such that the ultrasonic treatment is targeted to the target tissues/volume (such as, for example, the adipose tissue) and does not affect other tissues (such as, for example, muscles, bones, nerve tissue, and the like).

According to some embodiments, there is provided a device for automatic fat thickness measurement, the device may include an ultrasonic imager adapted to provide an ultrasonic image of a target area of a subject body and a processor unit adapted to process said image and to provide a measurement indicative of the thickness of fat layer in said target area.

According to additional embodiments, the measurements are adapted to be displayed to a caregiver who is operating the device for automatic fat thickness measurements. The measurements of the thickness of fat layer may include a numerical value. The numerical value may be in units of millimeters.

According to further embodiments, the device for automatic fat thickness measurements may further include an echo-signal sensor.

According to additional embodiments, the device may further be adapted to provide the fat thickness measurements to a controller of a transducer. The transducer may be adapted to transmit focused ultrasound to the target area. The focused ultrasound may be adapted to lyse fat tissue. The focused ultrasound may include high intensity focused ultrasound (HIFU), low intensity focused ultrasound (LIFU), or both.

According to further embodiments, the controller may be further adapted to adjust one or more parameters related to the transducer, according to the measurements provided by the device.

According to additional embodiments, the device may be associated with the transducer. The device may be integrated with the transducer. According to further embodiments, the device may be operated before the transducer is operated.

According to some embodiments, there is provided a system for lysing of adipose tissue that includes a fat measuring device adapted to automatically measure thickness of a fat layer in a target area of a subject, a transducer adapted to transmit focused ultrasound, and a controller adapted to receive a measurement from the fat measuring device and to trigger the transducer to transmit focused ultrasound to the fat layer.

According to some embodiments, the controller may be further adapted to adjust one or more parameters related to the transducer to facilitate the transmission of focused ultrasound to said fat layer.

According to additional embodiments, the fat measuring device may include an ultrasonic imager, an echo-signal sensor, a processing unit, or any combination thereof.

According to yet additional embodiments, the measurements are adapted to be displayed to a caregiver who is operating the system.

According to further embodiments, the thickness of the fat layer may include a numerical value. The numerical value may be in units of millimeters.

According to additional embodiments, the focused ultrasound is adapted to lyse fat tissue. The focused ultrasound may include high intensity focused ultrasound (HIFU), low intensity focused ultrasound (LIFU), or both.

According to some embodiments, the fat measuring device may be associated with the transducer. The fat measuring device may be integrated with said transducer.

According to yet further embodiments, the fat measuring device may be operated before the transducer is operated.

According to yet additional embodiments, the one or more parameters related to the transducer may include power, electrical voltage, focal distance, or any combination thereof.

According to further embodiments, the controller may further be adapted to issue an alert if the fat thickness measurements are not within the range of the focal region of the ultrasonic energy. The controller may further be adapted to adjust one or more parameters related to the transducer automatically, manually, or both.

According to some embodiments, there is provided a method for automatic measurement of fat thickness in a target area of a subject body that includes obtaining an ultrasonic image of the target area, analyzing the image to identify fat tissue in the target area and calculating fat tissue thickness in the target area. The fat tissue thickness may include a numerical value. The numerical value may be in units of millimeters. The method may further include displaying the calculated fat thickness.

According to further embodiments, the ultrasonic image of the method for automatic measurement of fat thickness may be obtained by an automatic fat measuring device. The automatic fat measuring device may include an ultrasonic imager, an echo-signal sensor, a processing unit, or any combination thereof.

According to additional embodiments, analyzing the image may include segmenting the image.

According to some embodiments, there is provided a method for lysing of adipose tissue that includes automatically measuring thickness of a fat layer in a target area of a subject and based at least partially on the measured thickness of the fat layer, transmitting focused ultrasound to the fat layer.

According to further embodiments, the method for lysing of adipose tissue may further include adjusting one or more parameters related to the transmission of focused ultrasound. The one or more parameters may include focal distance, electrical voltage, frequency, power, or any combination thereof. The focused ultrasound comprises high intensity focused ultrasound (HIFU), low intensity focused ultrasound (LIFU), or both.

According to further embodiments, in the method for lysing of adipose tissue, the thickness of fat layer may include a numerical value. The numerical value may be in units of millimeters. The method may further include displaying the numerical value.

According to additional embodiments, automatically measuring a thickness of a fat layer in the method for lysing of adipose tissue may include obtaining an ultrasonic image of the target area and analyzing the image to identify fat layer in the target area and determine thickness of the fat layer.

According to additional embodiments, automatically measuring the thickness of the fat layer in the method for lysing of adipose tissue may be performed by a fat measuring device. The fat measuring device may include an ultrasonic imager, an echo-signal sensor, a processing unit, or any combination thereof.

According to further embodiments, the method for lysing of adipose tissue may further include issuing an alert if the thickness of the fat layer is not within the focal region of the ultrasonic energy.

According to some embodiments, there is provided a method for dynamic modification of ultrasonic treatment that includes determination of fat tissue thickness in a target area of a subject body according to an ultrasonic image of the target area, and modifying one or more parameters related to an ultrasonic transducer that is adapted to provide ultrasonic energy to the target area, such that the ultrasonic energy is focused to the fat tissue. According to additional embodiments, the determination of fat tissue thickness may be performed automatically. The ultrasonic energy may include high intensity focused ultrasound (HIFU), low intensity focused ultrasound (LIFU), or both.

According to further embodiments, the one or more parameters related to the ultrasonic transducer, in the method for dynamic modification of ultrasonic treatment, may include focal distance, conductivity, electrical voltage, frequency, power or any combination thereof.

According to further embodiments, the ultrasonic transducer, in the method for dynamic modification of ultrasonic treatment, may include at least one transducing element.

According to additional embodiments, the dynamic modification may include modification in real time, during treatment.

In addition to the exemplary aspects and embodiments described above, further aspects and embodiments will become apparent by reference to the figures and by study of the following detailed descriptions.

### BRIEF DESCRIPTION OF THE FIGURES

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIGS. 1A-B—a schematic illustration of a cross sectional view of an exemplary ultrasonic transducing element being used to provide focused ultrasound to target tissue;

FIG. 2A—an illustration of a flow chart of a method of automatic fat thickness measurements, according to some embodiments;

FIG. 2B—illustration of exemplary results of a method of automatic fat measurements, according to some embodiments;

FIG. 3—schematic illustration of a block diagram of a fat measuring device, according to some embodiments;

FIG. 4—schematic illustration of a block diagram of a fat measuring device associated with a transducer, according to some embodiments;

FIG. 5—schematic illustration of a block diagram of a fat measuring device associated with a transducer, according to some embodiments;

FIG. 6A—schematic illustration of a system of automatic fat thickness and ultrasonic treatment, according to some embodiments;

FIG. 6B—illustration of a graphical user interface of a system of automatic fat thickness and ultrasonic treatment, according to some embodiments; and

FIGS. 7 A-C—schematic illustration of a perspective view of exemplary transducers.

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the embodiments may be practiced without specific details being presented herein. Furthermore, well-known features may be omitted or simplified in order not to obscure the embodiments.

As referred to herein, the term Focused Ultrasound (FU) is related to ultrasonic energy that may be externally and non-invasively applied to a surface in a focused manner such that the energy is focused to a specified internal target area. The ultrasonic (acoustic) energy applied may be, for example, in the form of waves. Applying and focusing the ultrasonic energy may be performed in various ways, such as, for example, by an ultrasonic transducer that may be adapted to focus the ultrasonic energy. The focused ultrasonic energy may include various frequency levels, such as in the range of 100 to 1500 kHz. For example, FU may include High Intensity Focused Ultrasound (known in the art as HIFU). For example, the FU may be applied to/on a subject skin and focused on a subcutaneous target area/volume, such as fat cells and adipose tissue.

As referred to herein, the term “HIFU” relates to High Intensity Focused Ultrasound—the use of high intensity focused ultrasound energy in ultrasound treatment (therapy). Ultrasound treatment may induce a vast range of biological effects at different exposure levels. At low levels, essentially reversible cellular effects can be produced, whereas at higher intensities, instantaneous cell death may occur. Accordingly, ultrasound therapies may be broadly divided into two groups: “high” power and “low” power therapies. At the one end of the spectrum, high power therapies include, for example, high intensity focused ultrasound (HIFU) and/or lithotripsy, while at the other end, low power therapies comprise, for example, sonophoresis, sonoporation, gene therapy and/or bone healing. According to some embodiments, the term HIFU may further encompass MIFU and/or LIFU.

As referred to herein, the term “MIFU” relates to Mid Intensity Focused Ultrasound—the use of medium intensity focused ultrasound energy in ultrasound treatment.

As referred to herein, the term “LIFU” relates to Low Intensity Focused Ultrasound—the use of low intensity focused ultrasound energy in ultrasound treatment.

As referred to herein, the term “subject” may relate to whom the ultrasonic energy is applied. Preferably, a “subject” is a patient who is being treated by the ultrasonic energy.

As referred to herein, the terms “treatment provider”, “operating personnel”, “caregiver”, “provider” and “care provider” may interchangeably be used. The terms relate to a person who may treat and/or monitor the subject. For example, a caregiver may include nurse, physician, therapist, practitioner, and the like.

As referred to herein the term “FMD” relates to a fat measuring device.

As referred to herein, the term “AFTM” relates to automatic fat thickness measurement(s).

As referred to herein, the terms “procedure” and “treatment” may interchangeably be used.

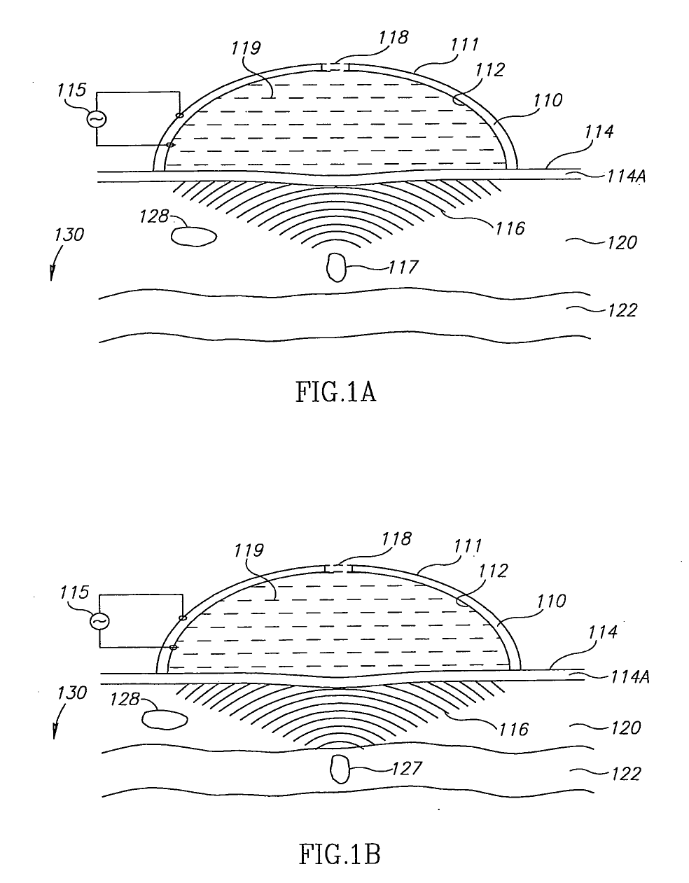

Reference is now made to FIG. 1A, which schematically illustrates a cross sectional view of an exemplary ultrasonic hemi-spherically shaped focusing transducing element 110, typically being used to provide focused ultrasound (in the range of high to mid to low intensity focused ultrasound (HIFU, MIFU, LIFU, respectively)) to lyse fat tissue 120 in a tissue region of a subject's body below the subject's skin 114. Also illustrated are exemplary neighboring tissues, such as, for example, muscle tissue 122, blood vessels 128 and skin tissue 114A. Nerve tissue and connective tissue are omitted from the illustration. The transducing element 100 may be produced using any of various methods and devices known in the art, and is formed having electrodes 111, 112, in the form of thin conducting coatings on its surfaces. The transducing element is driven by means of, for example, a high frequency power source 115, which applies a voltage between the electrodes 111, 112, of the transducer, thus exciting resonant vibration modes of the transducing element, and generating high intensity ultrasound waves for killing, damaging or destroying target tissue, such as, for example, fat (adipose) tissue. The transducing element is optionally filled with a suitable coupling medium 119 for acoustically coupling the transducing element to the subject's skin surface 114. The transducer may further include an aperture, such as, for example aperture 118. Due to the concave shape of the transducing element, the ultrasound waves (116) may be focused towards a focal region (117). Focal region 117 is generally in the form of an ellipsoid, having its major axis along the wave propagation direction (130). The size, location and depth of this focused region are dependent on a number of factors, such as, for example, the curvature of the transducing element, the frequency of ultrasound emitted, and the like. In order to ensure maximal efficiency and accuracy of the lysis of the fat (adipose) tissue, and to further increase safety of the fat lysis treatment, the ultrasound energy should be focused to the fat tissue, and not to neighboring tissue, so as to avoid damage to neighboring tissues that may be caused by the ultrasonic energy. Hence, the deposition of the focal region should be confined to the fat tissue and not to neighboring tissues, as illustrated, for example, in FIG. 1A, wherein focal region 117 is deposited in fat tissue 120 and does not affect neighboring tissues. FIG. 1B illustrates an undesired treatment event wherein focal region 127 is deposited in neighboring tissue (such as, for example, muscle tissue 122) and not to the desired area of fat tissue 120. In such an event, the result of the ultrasonic treatment may include an undesirable effect, wherein the fat tissue is not affected by the treatment, while neighboring tissue, which should not be affected by the focused ultrasonic energy, may be damaged due to the interaction of the focused ultrasonic energy with the tissue.

Interaction of the focused ultrasound waves with the tissue on which they are focused is dependent on a number of factors: thermal effects, which usually result in coagulation of the tissue, and are non-selective, the acoustic energy affects whichever tissue it encounters; Rupture or mechanical effects, which tear the cell walls, thus damaging the cell structure itself. This may not destroy the cell immediately, but may damage it sufficiently that it dies within a period following the treatment. This may be in the range of hours or days, depending on the extent and type of damage inflicted. This phenomenon is generally highly selective with regard to the type of tissue on which the ultrasound impinges, but it requires a high enough level of energy on target to be effective. Such mechanical effects may include streaming, shear or tensional forces; and cavitation effects, in which small bubbles are formed within the tissue. Therefore, in order to ensure maximal efficacy and accuracy of the ultrasonic treatment, and to provide the highest safety levels, it is desirable that the ultrasonic waves are focused to the fat tissue (as illustrated, for example, in FIG. 1A), and thus limit undesired side effects on other neighboring (surrounding) tissues, such as skin tissue, connective tissue, muscle tissue, nerve tissue, blood vessels, and the like. To this aim, distinguishing the fat tissue from other surrounding tissue should be performed and furthermore, the thickness of the fat tissue should be determined as accurately as possible, in order to ensure that the ultrasonic waves are focused as much as possible to the desired target tissue, at a desired depth of treatment, and thus would have minimal undesired effects on neighboring tissues.

According to some embodiments, there is thus provided an automatic fat thickness measuring device, method and system that may provide a caregiver with real-time measurements indicative of the fat thickness in the target area to be treated. The fat measuring device may further be adapted to determine fat thickness in the target area, by implementing the method for automatic fat thickness measurements. The fat measuring device may further be associated with an ultrasonic transducer that is adapted to provide ultrasonic energy to a target area of a subject. The fat measuring device may further interact with the controller system of the ultrasonic transducer to optionally adjust one or more parameters related to the ultrasonic transducer, so as to allow the caregiver to use the ultrasonic transducer in a body contouring procedure, such that the procedure is performed at the most accurate and effective depth of treatment, such that fat tissue is specifically targeted by the ultrasonic treatment. The parameters related to the ultrasonic transducer may include, for example, operating parameters of the ultrasonic transducer, the mode of operation of the ultrasonic transducer, and the like and may include such parameters as, but not limited to: power, electrical voltage, focal distance, or any combination thereof.

According to some embodiments, the fat thickness measuring may include an automatic fat thickness measurement (referred to herein as “AFTM”), that may allow the automatic measurement and determination of the thickness of the fat layer (tissue) of the subject being treated. Determination of the fat thickness may be performed, for example, by analyzing an ultrasonic image of the designated target (treatment) area, to distinguish the fat tissue from other neighboring tissues and to determine the thickness of the fat tissues. Obtaining the ultrasonic image may be performed by any method known in the art of obtaining ultrasonic images, such that, according to the reflected pattern of the ultrasonic waves from a target region, an image of the region may be constructed. The automatic determination of the fat layer from other surrounding tissues, and the calculation of the thickness of the fat tissue, may be performed by a method of image analysis, for example, by use of one or more algorithms that may be used to distinguish various neighboring tissues from the fat tissue, and once fat tissue has been determined, the thickness of the fat tissue may further be calculated. According to additional embodiments, the method of AFTM may further include a method to dynamically modify the ultrasonic treatment related to the AFTM. For example, the ultrasonic treatment may include lysing adipose tissue by use of focused ultrasonic energy that is directed towards fat tissue. The method may thus allow dynamic, real time modification of parameters associated with said treatment, in accordance with the AFTM results.

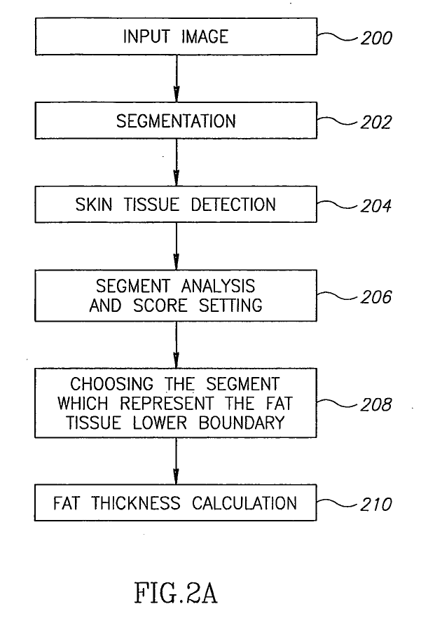

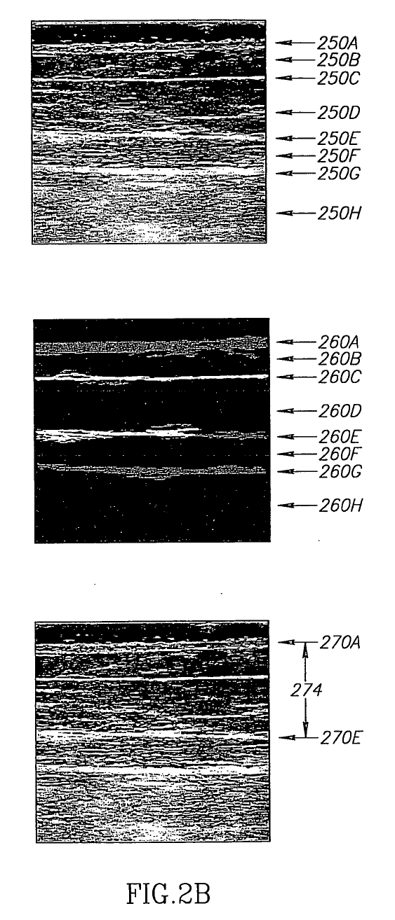

Reference is now made to FIG. 2A, which illustrates a flow chart of an algorithm that may be used for AFTM, according to some embodiments. At step 200, an input image is obtained, as further detailed hereinbelow. At the next step, 202, the image is being segmented to various segments, starting from the uppermost layer of the image and going downwards toward the internal tissues. At step 204, the skin tissue layer, which is the uppermost layer, is determined according to the segmentation performed at step 202. At step 206 the various segments of step 202 are being analyzed. Analysis of the various segments is performed by providing measurements related to each of the segments. The measurements may include such measurements as length, width, slenderness, mean brightness and the Y-location (depth relatively to the skin) of the center of the segment. According to measurements of the various segments, each segment is given a score, which is indicative of the probability of the respective segment to represent a fat tissue. In step 208, the segment that represents the fat tissue lower boundary, that is, the deepest fat region that has been identified in the image (the most distant fat layer from the skin), is being chosen. In step 210, the thickness of the fat layer is being determined by calculating the distance between the uppermost fat layer detected (that is the fat layer that is closest to the skin) and the lower fat tissue boundary. The thickness may be measured (and presented, if need be) in units of distance, such as, for example, in units of millimeters (mm). In addition, any filter and/or filtering methods (such as smoothing filters, and the like) either known today or to be developed in the future, may also be used with the algorithm.

Reference is now made to FIG. 2B, which demonstrate exemplary results of a method of automatic fat measurement, according to some embodiments. Uppermost panel (I) of FIG. 2B demonstrates an exemplary ultrasonic image of a target region. Various structures and layers, such as layers 250A-250H may be identified. In this example, layer 250A is determined to be skin tissue, and layers 250B-E are determined to include fat tissue. Panel II of FIG. 2B illustrates the image of panel I, after it has been analyzed and segmented. Segments 260 A-H are illustrated. Segments 260A-H correspond to tissues layers 250A-H. Panel III of FIG. 2B illustrates tissue layer 270A, which has been determined to be the skin layer, and tissue layer 270E, which has been determined to be the lower boundary of the fat layer. The distance, 274, between the uppermost skin tissue (270A) and the lower boundary of the fat tissue (270E) is the calculated thickness of the fat layer. Moreover, prior knowledge regarding the expected tissue structure, as a function of the location of the target area on the body may also be incorporated to the method of automatic fat thickness measurements, in order to increase accuracy of the measurements. For example, prior knowledge may include fat tissues structure, expected muscle thickness, expected nerves distribution, expected blood vessel density, and the like, or any combination thereof. Comparison of automatic fat thickness measurements obtained by the method described hereinabove and manual fat thickness determination by manual (visual) examination of ultrasonic images by a caregiver, demonstrates that the automatic fat thickness measurements are at least as accurate as manual evaluation of the fat thickness by an expert care provider.

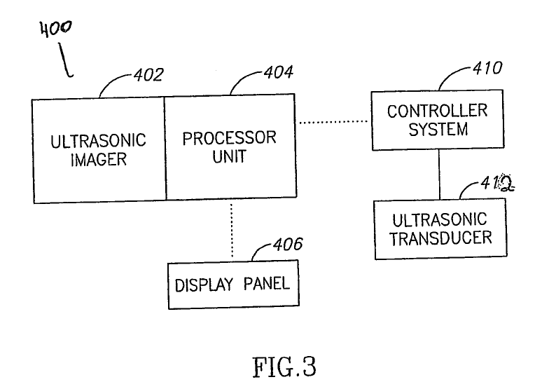

According to some embodiments, AFTM may be performed by a device, referred to herein as a “fat measuring device”. The fat measuring device (FMD) may include any type of ultrasonic imager that may be adapted to provide an image or a series of images (scan) of a target region and to further analyze said image and to compute the thickness of the fat layer in said image, for example, by use of an algorithm, such as the algorithm of FIG. 2A by a computing unit. The fat measuring device (FMD) may further include any type of echo-mode signal transducer and/or Doppler-effect imager. The FMD may be operatable by a caregiver. The fat measuring device may be further adapted to connect to the controller of an ultrasonic transducer and to provide the controller with the results that are indicative of the thickness of the fat layer. The connection between the FMD and the transducer may include any communication route, such as wires, cables, wireless, and the like. The FMD may be operated prior to the ultrasonic treatment. For example, the FMD may be used to scan/map the treatment area and to determine, within the treatment area, what is the thickness of the fat layer. The controller may automatically adjust the operating mode and/or operating parameters of the transducer, such that the modified operating parameters/operating mode are now in coordination with the fat thickness measurements. In addition or alternatively, the controller may present the results obtained from the fat measuring device to the caregiver, such that the caregiver may have an indication regarding the depth and thickness of the fat layer. The caregiver may then, if need be, change the operating parameters/operating mode of the controller (and hence, the transducer), such that the treatment efficiency is maximized. For example, if the operating parameters/operating mode of the transducer are such that the target region of the focused ultrasonic energy is at 14 mm, and the measurements of the FMD show that the fat thickness layer is determined to be at 10 mm, then the controller may change the operating mode and/or operating parameters of the transducer, and/or alert the caregiver regarding the measurements and allow the caregiver to choose his preferred mode of operation. For example, the controller may prevent the transducer from operating. For example, the controller may adjust the working parameters of the transducer. For example, by changing the power provided to the transducing element, the focal region of the focused energy may be changed, such that it is now at a depth of 9 mm and only the fat layer is targeted by the focused ultrasonic energy. For example, in the case of multiple transducing elements, selective operation of transducing elements may be performed such that the focal region of the focused energy may be changed, such that it is now at a depth of 9 mm and only the fat layer is targeted by the focused ultrasonic energy. Reference is now made to FIG. 3, which schematically illustrates a block diagram of a fat measuring device, according to some embodiments. As shown in FIG. 3, the fat measuring device, 400 includes an ultrasonic imager (402) adapted to obtain/acquire ultrasonic image of a target area of a subject body. Ultrasonic imager 402 may include any type of ultrasonic imager, such as, for example, an ultrasonic image scanner. Ultrasonic imager 402 may be manually operated by a caregiver. In addition, or alternatively, ultrasonic imager 402 may be operated automatically, after being positioned to a target area region. Fat measuring device 400 may further be associated (physically and/or functionally) with a processor unit (404). Association between ultrasonic imager 402 and processor unit 404 may include direct or indirect connection, permanent or transient connection, permanent or transient physical interaction, or any combination thereof. Processor unit 404 is adapted to analyze the image acquired by image scanner 402, by any of the methods described herein for automatic fat thickness measurements. Processor unit 404 is further adapted to provide the value of fat thickness measurements to the caregiver. Providing the value may be performed, for example, by displaying the value on optional display panel 406 which may be connected to the fat measuring device. In addition, or alternatively, the fat measuring device 400 may be associated (physically and/or functionally) and/or connected, for example, by wires, to a controller system (410) of an ultrasonic transducer (412), which is adapted to provide ultrasonic treatment to a subject body. The connection between fat measuring device 400 and controller system 410 may be used for the transfer of information between fat measuring device 400 and controller system 410 and may include any communication route, such as wires, cables, wireless, or any combination thereof.

According to some embodiments, the fat measuring device (FMD) may be associated with an ultrasonic transducer and may be adapted to provide real time measurements of the fat thickness, during treatment and/or before treatment. The FMD may be integrally associated with the transducer. The FMD may be transiently associated with the transducer. The FMD may be situated within the transducer, at any region relative to the transducing element. The FMD may be operable before the transducer is operative, to provide acoustic energy. The FMD may be operative simultaneously with the transducer. The FMD may include any type of ultrasonic imaging device that may be adapted to be associated with the transducer and to provide an image or a series of images (scan) of a target region and to further analyze said image and to compute the thickness of the fat layer in said image, by a computing unit. The fat measuring device (FMD) may further include any type of echo-mode signal transducer and/or Doppler-effect imager. The fat measuring device may be further adapted to directly or indirectly connect to the controller of the transducer and to provide the controller with the results of the computed thickness of the fat layer. The connection between the FMD and the transducer may include any communication route, such as wires, cables, wireless, and the like. The controller may accordingly automatically adjust the operating mode and/or operating parameters of the transducer, such that the modified parameters are now in coordination with the fat thickness measurements. In addition, or alternatively, the controller may present the results obtained from the fat measuring device to the caregiver, such that the caregiver may have an indication regarding the depth and thickness of the fat layer. The caregiver may then, if need be, change the operating parameters/operating mode of the controller (and hence, the transducer), such that the treatment efficiency is maximized. For example, if the operating parameters/operating mode of the transducer are such that the target region of the focused ultrasonic energy is at 14 mm, and the measurements of the FMD show that the fat thickness layer is determined to be at 10 mm, then the controller may change the operating mode and/or operating parameters of the transducer and/or alert the caregiver regarding the measurements and allow the caregiver to choose his preferred mode of operation. For example, the controller may prevent the transducer from operating. For example, the controller may adjust the working parameters of the transducer. For example, by changing the power provided to the transducing element, the focal region of the focused energy may be changed, such that it is now at a depth of 9 mm and only the fat layer is targeted by the focused ultrasonic energy. For example, in the case of multiple transducing elements, selective operation of transducing elements may be performed such that the focal region of the focused energy may be changed, such that it is now at a depth of 9 mm and only the fat layer is targeted by the focused ultrasonic energy.

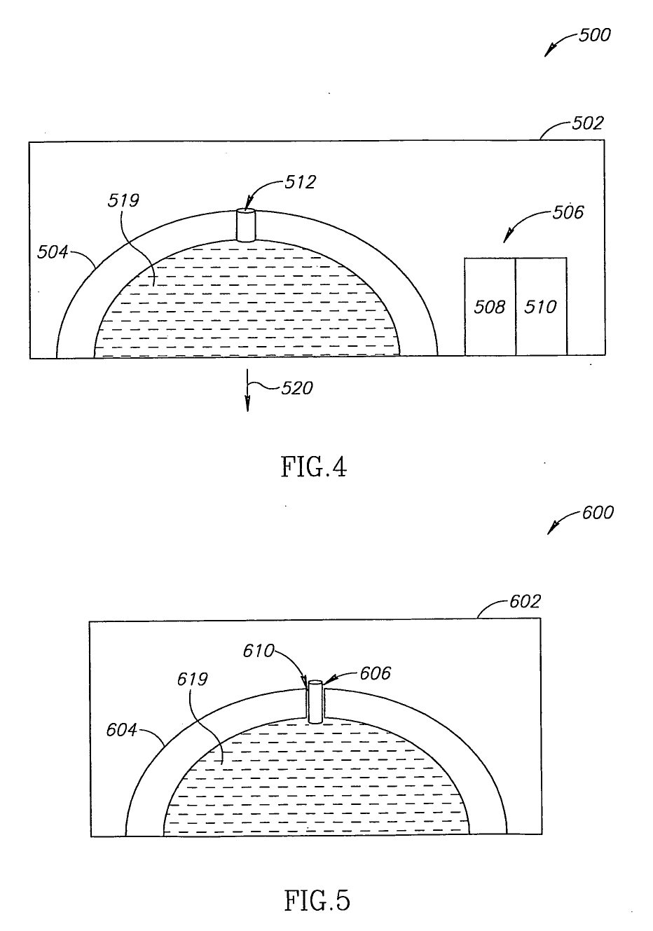

Reference is now made to FIG. 4, which illustrates a schematic cross sectional illustration of a fat measuring device associated with a transducer, according to some embodiments. As shown in FIG. 4, transducer 500 includes a casing (housing) 502. Within casing 502, one or more transducing elements (shown as one arched transducing element 504), are located. A round aperture (512) is located substantially at the center of the top of the outer (arched) surface of transducing element 504. The transducing element is optionally filled with a suitable coupling medium 519. In addition, a fat measuring device (506) is located within the casing of the transducer. Fat measuring device 506 is preferably located such that it is directed in the direction (520) of the axial propagation of the ultrasonic energy waves produced by the transducing element, and may be located at any orientation relative to the transducing element, such as, on the sides of the transducing element (as illustrated, for example, in FIG. 4), on top of the upper surface (concaved side) of the transducing element, below the lower surface (convex side) of the transducing element, or any combination thereof. Fat measuring device 506 may include sensor 508, which may include an ultrasonic imager, adapted to obtain/acquire ultrasonic image of a target area of a subject body, an echo-mode sensor, and the like. Fat measuring device 506 may optionally further include a processor unit (510). Processor unit 510 may be physically and/or functionally associated with sensor 508. Processor unit 510 is adapted to analyze the data acquired by sensor 508, by any of the methods described herein for automatic fat thickness measurements. For example, if sensor 508 includes an ultrasonic imager, processor unit 510 is adapted to analyze an image acquired by the ultrasonic imager. For example, if sensor 508 includes an echo-signal sensor, processor unit 510, is further adapted to analyze the echo signals of the echo-signal sensor and to accordingly, automatically determine fat thickness. Fat measuring device 506 may be adapted to operate in synchronization with transducing element 504, such that when the transducer is aimed towards the target area on the subject body, the fat measuring device may acquire an ultrasonic image of the target area, analyze the image and provide results to the controller of the transducer, to which the fat measuring device may be directly or indirectly connected. Alternatively, fat measuring device 506 may be adapted to operate in synchronization with transducing element 504, such that when the transducer is aimed towards the target area on the subject body, the fat measuring device may acquire an ultrasonic image or echo-mode signals of the target area and send the acquired data to the controller of the transducer, which may then process/analyze the data received from the fat measuring device to determined fat thickness. Only after measurements have been obtained by the fat measuring device may the transducing element be operable. Nevertheless, if the fat thickness measurement results indicate that the operating parameters/operating mode of the transducer do not match the fat thickness measurements, as explained hereinabove, the controller may not allow operation of the transducer until operating parameters/operating mode are adjusted accordingly.

According to some embodiments, the fat measuring device (FMD) may be associated with an ultrasonic transducer and may be adapted to provide real time measurements of the fat thickness, during treatment and/or before treatment. The FMD may be integrally associated with the transducer. The FMD may be transiently associated with the transducer. The FMD may be operable before the transducer is operative to provide acoustic energy. The FMD may be operative simultaneously with the transducer. The FMD may include any type of echo-signal sensor that may emit ultrasonic waves and analyze the pattern of the reflected waves, in order to provide assessment of the thickness of the fat layer. The fat measuring device may be further adapted to directly or indirectly connect to the controller of the transducer and to provide the controller with the results of the computed thickness of the fat layer. The connection between the FMD and the transducer may include any communication route, such as wires, cables, wireless, and the like. The controller may, accordingly, automatically adjust the operating mode and/or operating parameters of the transducer, such that the modified operating parameters/operating mode are now in coordination with the fat thickness measurements. In addition or alternatively, the controller may present the results obtained from the fat measuring device to the caregiver, such that the caregiver may have an indication regarding the depth and thickness of the fat layer. The caregiver may then, if need be, change the operating parameters/operating mode of the controller (and hence, the transducer), such that the treatment efficiency is maximized. For example, if the operating parameters/operating mode of the transducer are such that the target region of the focused ultrasonic energy is at 14 mm, and the measurements of the FMD show that the fat thickness layer is determined to be at 10 mm, then the controller may change the operating mode and/or operating parameters of the transducer and/or alert the caregiver regarding the measurements and allow the caregiver to choose his preferred mode of operation. For example, the controller may prevent the transducer from operating. For example, the controller may adjust the working parameters of the transducer. For example, by changing the power provided to the transducing element, the focal region of the focused energy may be changed such that it is now at a depth of 9 mm, and only the fat layer is targeted by the focused ultrasonic energy. For example, in the case of multiple transducing elements, selective operation of transducing elements may be performed such that the focal region of the focused energy may be changed such that it is now at a depth of 9 mm, and only the fat layer is targeted by the focused ultrasonic energy.

Reference is now made to FIG. 5, which illustrates a schematic block diagram cross sectional illustration of fat measuring device associated with a transducer, according to some embodiments. As shown in FIG. 5, transducer 600 includes a casing (602). Within casing 602, one or more transducing elements (shown as one arched transducing element, 604) are located. A round aperture (610) is located substantially at the center of the top of the outer (arched) surface of transducing element 604. The transducing element is optionally filled with a suitable coupling medium 619. In addition, a fat measuring device (606) is located within the casing of the transducer. Fat measuring device 606 is preferably located within aperture 610 of transducing element 604. Fat measuring device 606 may include a sensor, such as, for example, an ultrasonic echo-signal sensor, adapted to emit ultrasonic waves and to detected reflection of the emitted ultrasonic waves, an ultrasonic imager, adapted to obtain/acquire ultrasonic image of a target area of a subject body, and the like. Fat measuring device 606 may further optionally include a processor unit, adapted to analyze the signals received by the sensor. The signals may include, for example, echo signals of the echo-signal sensor or ultrasonic images of a target area, acquired by imager sensor. The processor unit may then automatically determine fat thickness based on the received signals. Alternatively, the signals obtained by the sensor may be transferred to the controller system (not shown) of the transducer. The processor unit of the controller system may be adapted to analyze the echo-signals, and, accordingly, determine the thickness of the fat layer, for example, by constructing an image of the tissue, and analyzing said image by any of the methods described herein for AFTM. Fat measuring device 606 may be adapted to operate in synchronization with the transducing element, such that when the transducer is aimed towards the target area on the subject body, the sensor of the fat measuring device may acquire signals (such as an ultrasonic image of the target area, echo-signals, and the like). The fat measuring device may further analyze the acquired data and provide results to the controller of the transducer, to which the fat measuring device may be directly or indirectly connected. Alternatively, fat measuring device 606 may be adapted to operate in synchronization with the transducer, such that when the transducer is aimed towards the target area on the subject body, the fat measuring device may acquire an ultrasonic image or echo-mode signals of the target area and send the acquired data to the controller of the transducer, which may then process/analyze the data received from the fat measuring device to determined fat thickness. Only after measurements have been obtained by the fat measuring device may the transducing element be operatable. Nevertheless, if the fat thickness measurements results indicate that the operating parameters/operating mode of the transducer do not match the fat thickness measurements, as explained hereinabove, the controller may not allow operation of the transducer until operating parameters/operating mode are adjusted accordingly.

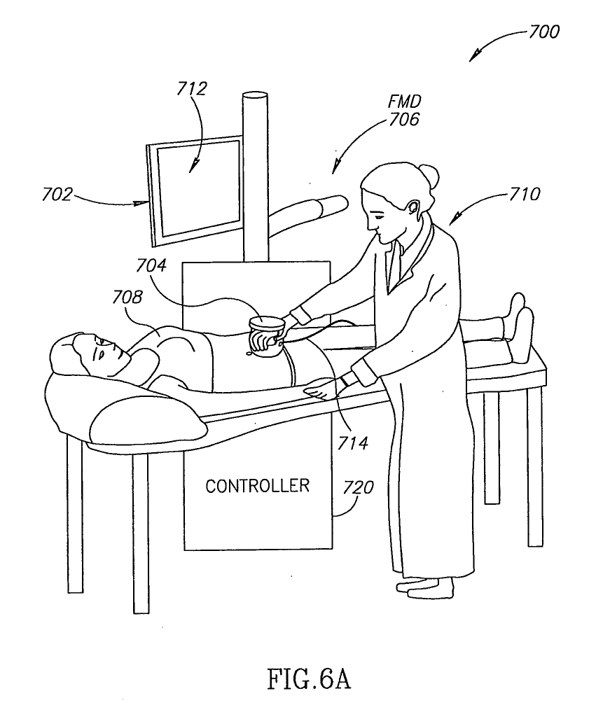

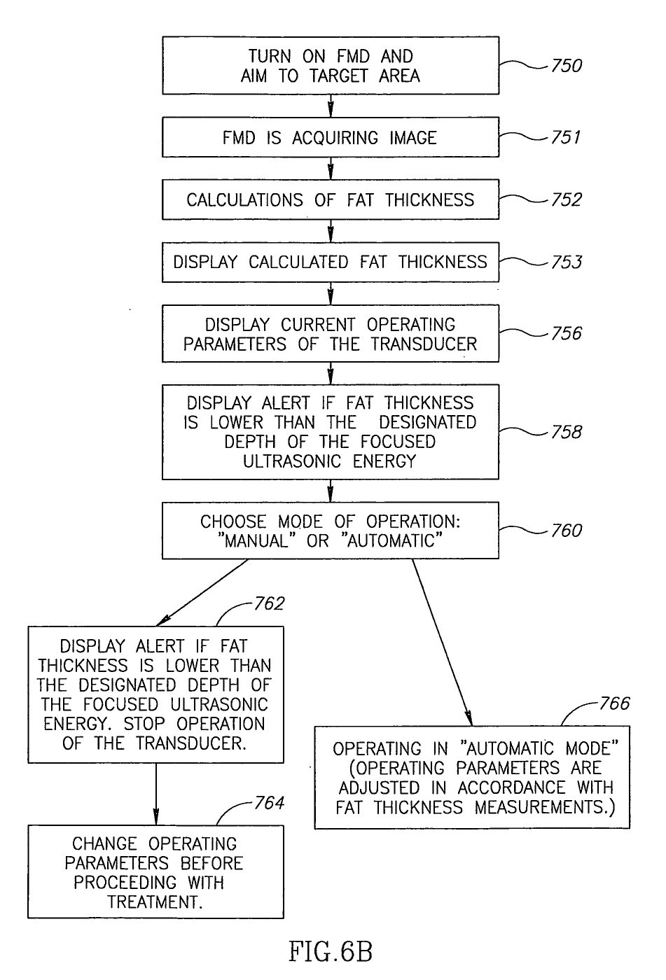

According to some embodiments, the FMD may be connected to a controller of a transducer. The FMD may be independently operated (such as illustrated, for example, in FIG. 3) and/or may be associated physically and/or functionally with the transducer (such as illustrated, for example, in FIGS. 4 and 5). The connection between the FMD and the controller may be direct or indirect and may include any type of connection, such as, by use of wires, cables, wireless, or any combination thereof. Reference is now made to FIG. 6A, which schematically illustrates a system of automatic fat thickness measurements and fat lysis by ultrasonic treatment, according to some embodiments. As shown in FIG. 6A, the system (700) may include a controller (720) and a transducer (704) that is connected to controller 720, for example, by cable 714. In addition, system 700 includes a fat measuring device, 706 (shown as an independent fat measuring device that is not physically associated with transducer 704). Also shown in FIG. 6A is a subject (708) and a caregiver (710). As shown in FIG. 6A, FMD 706 may be directly connected to controller 720, through connection 716. In the system shown in FIG. 6A, FMD 706 was already operated, and the results obtained by FMD 706 have already been transferred to controller 720. The results and/or user interface 712 (further detailed in FIG. 6B) may optionally be displayed to caregiver 710 on display panel (monitor 702), which is functionally associated with controller 720. According to the AFTM results provided by FMD 706, the operating parameters/operating mode of transducer 704 may be adjusted by controller 720, such that the ultrasonic energy emitted by the transducer is focused to a region which encompasses a fat layer, as determined according to the measurements provided by FMD 706.

Reference is now made to FIG. 6B, which illustrates a graphical user interface of an AFTM system, according to some embodiments. The graphical user interface described in FIG. 6 may be displayed, for example, on display panel (monitor 712) illustrated in FIG. 6A. As shown in stage 750, a message prompting to turn on the FMD and to aim the FMD towards the target area on the subject body (the area that is to be treated), is displayed. After the FMD is turned on, a message indicating that an image of the target area of the subject is being obtained is displayed in stage 751. In stage 752, a message indicates that calculations of the measurement of the fat thickness are being performed by FMD 706. In stage 753, the value of the fat thickness measurements is displayed as a numerical value in units of mm. In stage 756, the fat thickness measurement value is presented alongside the current operating parameters/operating mode of the transducer, including the current focal region of the depth of the focused ultrasonic energy to be produced by the transducer. At optional stage 758, an alert is displayed if the value of the fat thickness is lower than the designated depth of the focused ultrasonic energy. At stage 760, the caregiver is prompted to choose mode of operation—automatic or manual. If “manual” mode of operation is chosen, and the value of the fat thickness is not lower than the designated depth of the focused ultrasonic energy, the care provider may operate the transducer using the current operating parameters/operating mode. If “manual” mode of operation is chosen, and the value of the fat thickness is lower than the designated depth of the focused ultrasonic energy, at stage 762 an appropriate alert is issued to the caregiver and the controller to prevent the operation of the transducer. At stage 764, the caregiver is prompted to change operating parameters/mode of operation before proceeding with treatment. If “automatic” mode of operation is chosen at stage 760, the controller automatically compares the value of the fat thickness to the designated depth of the focused ultrasonic energy. If the value of the fat thickness is not lower than the designated depth of the focused ultrasonic energy, the transducer is operated using the current operating parameters/operating mode. If the value of the fat thickness is lower than the designated depth of the focused ultrasonic energy, the controller adjusts the operating parameters/operating mode of the transducer such that it is operated in accordance with the value of the fat thickness, and an appropriate message, as shown in stage 766 is prompted to the caregiver.

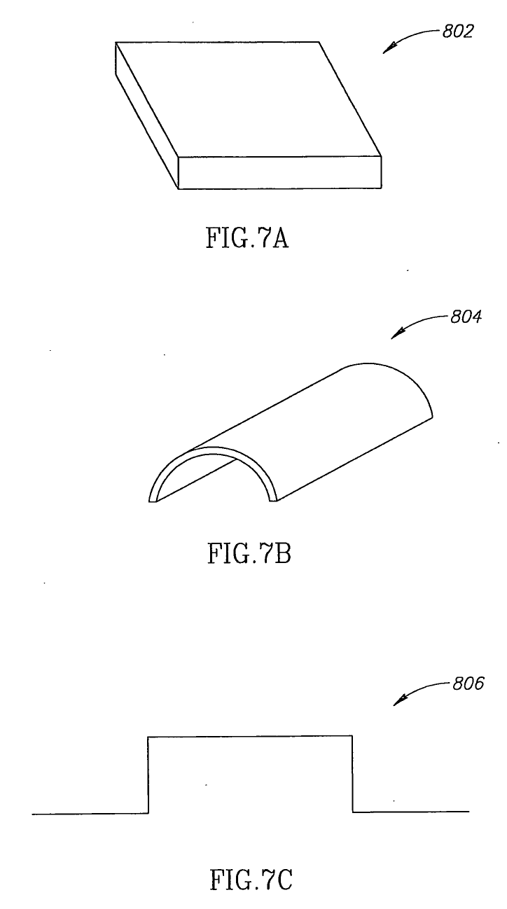

According to some embodiments, the ultrasonic transducer may include any type of ultrasonic transducer that may be adapted to provide acoustic energy to a target and/or a target area, which may include a target area of a subject body. The ultrasonic transducer may include any desired size and geometrical shape. Reference is now made to FIG. 7, which schematically illustrates perspective views of exemplary transducers. For example, as shown in FIG. 7A, transducer 802 is a flat, rectangular transducer. For example, as shown in FIG. 7B, transducer 804 is a half cylindrical transducer. For example, as shown in FIG. 7C, transducer 806 is a half cubical transducer. The ultrasonic transducer and the acoustic energy may be used in therapeutic procedures, cosmetic procedures, body contouring procedures, and the like. The target may include one or more tissues of a subject being treated by the acoustic energy. For example, the target may include any tissue and/or cell type of the subject, such as, for example, fat tissue, adipose tissue, cellulite tissue, and the like, or any combination thereof. The ultrasonic transducer may be used, for example in non-invasive body contouring procedures. The ultrasonic energy delivered by the transducer may include various forms, such as, for example, the energy may include Focused energy (FU), High Intensity Focused Ultrasound (known in the art as HIFU), Mid Intensity Focused ultrasound (MIFU), Surface energy (SU) or any combination thereof.

According to further embodiments, the ultrasonic transducer may include any type of ultrasonic transducer that may include one or more transducing elements, which are the elements that produce the acoustic energy. The transducing element may be constructed of materials such as metal, ceramics, Lead Zirconate Titanate (PZT), and the like. The transducing element may be flat, concave, convex or otherwise shaped. The thickness of the transducing element may be identical throughout or may be different along various regions of the transducing element. For example, For example, the thickness of the element may vary in the range of 0.1 mm to 10 mm. As a result of electrical energy provided to the transducing element, the element may vibrate and, as a result, produce acoustic energy. The electrical energy may be provided continuously and a continuous acoustic wave may be produced. The electrical power provided to the transducing element may be provided in pulses/nodes, and the resulting vibration energy produced by the vibration element may be provided in bursts. For example, the transducing element may vibrate at a resonance frequency in the range of about 100 to 1500 kHz. In general, the shape, size, thickness, composition and spatial location of the transducing element(s) may be adjusted so as to produce a requested acoustic energy and to target said energy to a desired target area.

According to additional embodiments, the ultrasonic transducer may include more than one piezoelectric element that may be used to produce acoustic waves in response to electrical energy stimulation. For example, the transducer may include one or more transducing elements (such as piezoelectric elements), that are connected and/or associated and/or adhered to each other and may operate synchronically or non-synchronically. For example, the transducer may include a transducing element that may be sectorized, wherein various regions of the transducing element may possess different properties. The one or more transducing elements may include one or more separable electrical connections (electrodes). The transducing elements(s) may further include one or more resonators, which are devices that exhibit acoustic resonance behavior (meaning it may oscillate at some frequencies with greater amplitude that at other frequencies). The resonator may be used as a wave-guide to guide ultrasonic waves in a desired pattern, direction, and the like

Operation of the transducer may be controlled by a controller that may provide the transducer with, for example, power, energy, fluids, software instruction, control and feedback information, and the like. The controller may determine the parameters according to which the transducer may operate. The controller may include several subunits that may be independent and/or interconnected and may be individually or commonly controlled. The controller may include a controlling unit that may include any appropriate hardware and software that may be used to control and coordinate operation of the transducer. For example, the controlling unit may include electronic circuits, processors, ROM and RAM memories, and the like. The controller may receive and send information to and from the transducer. The controller may include several power sources. For example, a high power pulser may provide power to the transducer (and in particular to the transducing element). Preferably, the operating parameters/operating mode set by the controller may be such that the transducer may operate in a mode wherein the effect of the energy produced by the transducer on the target area is maximized, with as low as possible undesired side effects (such as, for example, pain sensation, unwanted damage to neighboring tissues, and the like).

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

What we claim is:

1. A device for automatic fat thickness measurement, the device comprising:
an ultrasonic imager adapted to provide ultrasonic image of a target area of a subject body; and
a processor unit adapted to process said image and to provide a measurement indicative of the thickness of fat layer in said target area.

2. The device of claim 1, wherein said measurements are adapted to be displayed to a caregiver who is operating said device.

3. The device of claim 1, wherein said measurements of the thickness of fat layer comprises a numerical value.

4. The device of claim 3, wherein said numerical value is in units of millimeters.

5. The device of claim 1, further comprising an echo-signal sensor.

6. The device of claim 1, further adapted to provide said fat thickness measurements to a controller of a transducer.

7. The device of claim 6, wherein said transducer is adapted to transmit focused ultrasound to said target area.

8. The device of claim 7, wherein said focused ultrasound is adapted to lyse fat tissue.

9. The device of claim 8, wherein said focused ultrasound comprises high intensity focused ultrasound (HIFU), low intensity focused ultrasound (LIFU), or both.

10. The device of claim 6, wherein said controller is further adapted to adjust one or more parameters related to the transducer, according to the measurements provided by said device.

11. The device of claim 6 wherein said device is associated with the transducer.

12. The device of claim 6, wherein said device is integrated with said transducer.

13. The device of claim 6, wherein said device is operated before the transducer is operated.

14. A system for lysing of adipose tissue comprising:
a fat measuring device adapted to automatically measure thickness of a fat layer in a target area of a subject;
a transducer adapted to transmit focused ultrasound; and
a controller adapted to receive a measurement from said fat measuring device and to trigger said transducer to transmit focused ultrasound to said fat layer.

15. The system of claim 14, wherein said controller is further adapted to adjust one or more parameters related to the transducer to facilitate the transmission of focused ultrasound to said fat layer.

16. The system of claim 14, wherein said one or more parameters comprises power, electrical voltage, focal distance, or any combination thereof.

17. The system of claim 14, wherein said fat measuring device comprises an ultrasonic imager, an echo-signal sensor, a processing unit, or any combination thereof.

18. The system of claim 14, wherein said measurements are adapted to be displayed to a caregiver who is operating said system.

19. The system of claim 14, wherein said thickness of fat layer comprises a numerical value.

20. The system of claim 18, wherein said numerical value is in units of millimeters.

21. The system of claim 14, wherein said focused ultrasound is adapted to lyse fat tissue.

22. The system of claim 14, wherein said focused ultrasound comprises high intensity focused ultrasound (HIFU), low intensity focused ultrasound (LIFU), or both.

23. The system of claim 14, wherein said fat measuring device is associated with the transducer.

24. The system of claim 14, wherein said fat measuring device is integrated with said transducer.

25. The system of claim 14, wherein said fat measuring device is operated before the transducer is operated.

26. The system of claim 14, wherein said controller is further adapted to issue an alert if the fat thickness measurements are not within the range of the focal region of the ultrasonic energy.

27. The system of claim 14, wherein said controller is adapted to adjust one or more parameters related to the transducer automatically, manually, or both.

28. A method for automatic measurements of fat thickness in a target area of a subject body comprising:
obtaining an ultrasonic image of said target area;
analyzing said image to identify fat tissue in said target area; and
calculating fat tissue thickness in said target area.

29. The method of claim 28, wherein said fat tissue thickness comprises a numerical value.

30. The method of claim 29, wherein said numerical value is in units of millimeters.

31. The method of claim 28, wherein said ultrasonic image is obtained by an automatic fat measuring device.

32. The method of claim 31, wherein said automatic fat measuring device comprises an ultrasonic imager, an echo-signal sensor, a processing unit, or any combination thereof.

33. The method of claim 28, further comprising displaying said calculated fat thickness.

34. The method of claim 28, wherein said analyzing comprises segmenting said image.

35. A method for lysing of adipose tissue comprising:
automatically measuring a thickness of a fat layer in a target area of a subject; and
based at least partially on the measured thickness of the fat layer, transmitting focused ultrasound to the fat layer.

36. The method of claim 35, further comprising adjusting one or more parameters related to the transmission of focused ultrasound.

37. The method of claim 35, wherein said one or more parameters comprise focal distance, electrical voltage, frequency, power, or any combination thereof.

38. The method of claim 35, wherein said focused ultrasound comprises high intensity focused ultrasound (HIFU), low intensity focused ultrasound (LIFU), or both.

39. The method of claim 35, wherein said thickness of fat layer comprises a numerical value.

40. The method of claim 39, wherein said numerical value is in units of millimeters.

41. The method of claim 39, further comprising displaying said numerical value.

42. The method of claim 35, wherein said automatically measuring a thickness of a fat layer comprises obtaining an ultrasonic image of said target area and analyzing said image to identify fat layer in said target area and determining thickness of said fat layer.

43. The method of claim 35, wherein said automatically measuring a thickness of a fat layer is performed by a fat measuring device.

44. The method of claim 43, wherein said fat measuring device comprises an ultrasonic imager, an echo-signal sensor, a processing unit, or any combination thereof.

45. The method of claim 35, further comprising issuing an alert if the thickness of the fat layer is not within the focal region of the ultrasonic energy.

46. A method for dynamic modification of ultrasonic treatment comprising:
determination of fat tissue thickness in a target area of a subject body according to ultrasonic image of said target area; and
modifying one or more parameters related to an ultrasonic transducer that is adapted to provide ultrasonic energy to said target area, such that said ultrasonic energy is focused to said fat tissue.

47. The method of claim 46, wherein said determination of fat tissue thickness is performed automatically.

48. The method of claim 46, wherein said ultrasonic energy comprises high intensity focused ultrasound (HIFU), low intensity focused ultrasound (LIFU), or both.

49. The method of claim 46, wherein said one or more parameters comprises focal distance, conductivity, electrical voltage, frequency, power, or any combination thereof.

50. The method of claim 46, wherein said ultrasonic transducer comprises at least one transducing element.

51. The method of claim 46, wherein said dynamic modification comprises modification in real time, during treatment.
